(19) United States
(12) Patent Application Publication
(10) Pub. No.: US 2009/0186408 A1
(43) Pub. Date: Jul. 23, 2009

(54) Biocompatible bilayer porous matrix and preparation thereof

(75) Inventors: Tzu-Wei Wang, Taipei (TW); Yi-Chau Huang, Taipei (TW); Feng-Huei Lin, Taipei (TW)

Correspondence Address: BACON & THOMAS, PLLC, 625 SLATERS LANE, FOURTH FLOOR, ALEXANDRIA, VA, 22314-1176, US

(73) Assignee: National Taiwan University, Taipei (TW)

(21) Appl. No.: 12/010,014

(22) Filed: Jan. 18, 2008

(51) Int. Cl. C12N 5/02

(52) U.S. Cl. 435/373; 435/404

(57) ABSTRACT

The present invention provides a biocompatible bilayer porous matrix and preparation thereof. The bilayer porous matrix is composed of gelatin, chondroitin 6 sulfate, and hyaluronic acid, also, prepared through freeze-drying technique at different temperature and time duration to form varied pore sizes on each layer. The present invention also provides a method of cell culture using the bilayer porous matrix.

### BACKGROUND OF THE INVENTION

1. Field of the Invention

The present invention relates to a biocompatible bilayer porous matrix, more particularly to a porous matrix used for cell culture.

2. Description of the Prior Art

Clinically, the burned patient with large-area burn shall be saved by the skin graft operation, or the rehabilitation after operation shall be conducted. Theoretically, it is hoped that the patient can use his own normal skin for autograft, in order to resume the function of wound skin, and avoid other side effects caused by the graft. However, during the therapy process, the major problem is the normal skin for autograft is very limited, and new wound will be generated for the patient, so it is necessary to use other auxiliary materials.

At present, there are three methods to substitute the autograft of skin, such as the allograft by using the skin of dead body, the xenograft by using the skin of animal, and the method by using the artificial synthetic dressing (namely artificial skin). But these methods can be used as temporary protection only, the epidermis of autograft has to be used for the replacement later. In addition, the skin of allograft or xenograft will usually be rejected by the autoimmune function of patient, and infected by the germ, or the inflammation of patient may be induced. So it can be used as the temporary wound dressing only, and it has to rely on the medicine to control the immune rejection. Clinically, the allograft or artificial skin is usually used to protect the wound from the bacterial infection or avoid the loss of body fluid during the convalescent time of patient. After the dermis layer of patient is recovered, the artificial skin can be removed, and the formal autograft can be conducted. These methods are standard therapy procedure to treat the burned patient with large-area burn, but it is very expensive, and the operation is labor intensive.

The research of tissue engineering provides a direction of solving the above-mentioned problems. At present, there are a lot of researches associated with large-scale in vitro keratinocyte culture. But most of these cell culture substances only have monolayer epidermal structure, which lack the support of the connective tissue, and do not possess the elasticity or mechanical property of skin. Other in vitro cell culture methods only increase the fibroblast, but because the characteristics of different skin cells are quite different, different environment is required for the growth of cultivated cell, so there is still no artificial skin substitute with satisfactory effect.

Upon inquiring the patent database of Taiwan Intellectual Property Office, the Taiwanese Patent No. I265035 “Type II Collagen/Glycosaminoglycan/Hyaluronic Acid Porous Carrier And Preparation Thereof” disclosed a porous carrier used in the tissue engineering. But the prepared porous carrier is used for the cartilage cell culture, which is only used for the transplant substitute of cartilage tissue, and type II collagen can not meet the skin demand of burned patient. The dermis of skin is composed of type I collagen, not composed of type II collagen.

In order to overcome the foresaid drawbacks in the prior arts, the present invention provides a biocompatible bilayer porous matrix.

### SUMMARY OF THE INVENTION

Therefore, the present invention uses the tissue-engineering technique to develop a skin-like equivalent for promoting the repair and regeneration of the skin, in order to help the patient who requires the skin graft operation for large-area burn, or the wound is difficult to be closed, i.e. diabetes foot ulcers.

It is an aspect of the present invention to provide a biocompatible bilayer porous matrix, including a first porous matrix and a second porous matrix. The first porous matrix and the second porous matrix are composed of cross-linked gelatin, chondroitin 6 sulfate, and hyaluronic acid.

Preferably, the pore size of the first porous matrix is 10 to 50 μm, and the matrix thickness is 80 to 120 μm thereof, and the pore size of the second porous matrix is 50 to 180 μm, and the matrix thickness is 500 to 900 μm thereof.

More preferably, the pore size is 20 to 40 μm, and the matrix thickness is 90 to 110 μm for the first porous matrix.

More preferably, the pore size is 75 to 150 μm, and the matrix thickness is 600 to 800 μm for the second porous matrix.

It is another aspect of the present invention to provide a biocompatible bilayer porous matrix and preparation thereof, its steps include: first, (a) prepare an aqueous solution with gelatin, chondroitin 6 sulfate, and hyaluronic acid; (b) pour the aqueous solution into a mold, and freeze it quickly to form a first porous matrix; then, (c) apply the aqueous solution on the surface of the first porous matrix, and freeze it slowly to form a second porous matrix; and finally, (d) add a cross-linking agent to initiate the cross-linkage of the porous matrixes.

According to a preferred embodiment of the present invention, the preparation of the aqueous solution is to dissolve gelatin in water at room temperature, and then add chondroitin 6 sulfate and hyaluronic acid, respectively.

Preferably, the composition of the aqueous solution is 5 to 10 wt % of gelatin, 0.5 to 2.5 wt % of chondroitin 6 sulfate, and 0.3 to 0.5 wt % of hyaluronic acid.

According to another preferred embodiment of the present invention, the porous matrixes are fabricated through different freezing temperatures and time durations to form the pores with different size and density.

Preferably, the aqueous solution is frozen quickly to the temperature of −196° C. for 1 to 2 min to form the first porous matrix layer.

Preferably, the aqueous solution is frozen to the temperature of −80° C. for 180 min to form the second porous matrix layer.

According to another preferred embodiment of the present invention, the step (c) further includes step (c1): lyophilize the porous matrix at −70° C.

According to another preferred embodiment of the present invention, the cross-linking agent is carbodiimide.

Preferably, the cross-linking agent is 0.5 to 1 wt % of 1-ethyl-3-(3-dimethylaminopropyl) carbodiimide (EDAC).

Preferably, the cross-linking agent is 0.25 wt % of N-hydroxysuccinimide solution, which is reacted at pH 5.75 and 4° C.

According to another preferred embodiment of the present invention, after the cross-linking reaction, the porous matrix is rinsed by disodium phosphate solution to remove residual carbodiimide.

Preferably, after rinsing, the porous matrix is frozen at −80° C. followed by lyophilizing at −70° C.

It is another aspect of the present invention to provide a method of animal cell culture using the bilayer porous matrix according to the present invention, wherein the first porous matrix and the second porous matrix are used to cultivate different cells, respectively.

According to a preferred embodiment of the present invention, the first porous matrix is used to cultivate epidermal keratinocytes, and the second porous matrix is used to cultivate dermal fibroblasts.

Preferably, the animal cell is human cell.

The term “matrix” described in the present invention is also called a “substrate” or “scaffold”, which is used for cell growth and adhesion, and it mainly imitates the composition and content of extracellular matrix (ECM) of animals.

The term “gelatin” described in the present invention means a collagen molecule or fragment without three-dimensional structures, which can be refined or synthesized from animal tissue. The molecular structure of gelatin still reserves the domain for cell contact, proliferation, or differentiation. In addition, the gelatin is a biocompatible, nontoxic material, which is used for wound therapy widely.

The term chondroitin 6 sulfate (C6S) described in the present invention means the molecule derived from hexuronic acid (vitamin C) and hexosamine. Upon adding chondroitin 6 sulfate into gelatin solution, it can increase the resistance of gelatin solution to collagenase, improve the stability of structure, and increase the elasticity and porosity. The addition of chondroitin 6 sulfate can also improve the construction of skin basal membrane.

The term hyaluronic acid (HA) described in the present invention is a transparent colloid substance found in the connective tissue and dermal layer commonly. It is a polysaccharide composed of Glucuronic acid and N-acetylglucosamine. The hyaluronic acid possesses many characteristics such as biological absorbability, biocompatibility, viscous property, water retaining property etc., which has already been used in biomedical application generally.

The present invention provides a bilayer porous matrix and preparation thereof, which is fabricated successfully using different freezing temperatures and followed by lyophilization. It can be used as a scaffold of artificial skin for cell culture. In the first porous matrix (upper layer, prepared at −196° C.), the pore is smaller and denser, the preferred pore size is 20 to 40 μm, and the porosity is 30 to 40%, which is suitable for the attachment and proliferation of epidermal keratinocytes. In the second porous matrix (lower layer, prepared at −80° C.), the pore is larger and sparse, the preferred pore size is 75 to 150 μm, and the porosity is 70 to 80%, which is suitable for the migration and growth of dermal fibroblasts. The interconnected pores between two layers can provide the interaction opportunities for released cytokines and growth factors by dermal fibroblasts and epidermal keratinocytes, to accelerate the growth and differentiation process of skin tissues.

The above aspects and advantages of the present invention will become more readily apparent to those ordinarily skilled in the art after reviewing the following detailed description and accompanying drawings, in which:.

### BRIEF DESCRIPTION OF THE DRAWINGS

FIG. 1 is a flowchart schematically illustrating the preparation for bilayer porous matrix according to the present invention;

FIG. 2 is diagram schematically illustrating the structure for bilayer porous matrix according to the present invention; and

FIG. 3 is a diagram schematically illustrating the scanning electron microscopy image for bilayer porous matrix according to the present invention.

### DESCRIPTION OF THE PREFERRED EMBODIMENT

The invention is described more specifically with reference to the following embodiments. It is to be noted that the following descriptions of preferred embodiments of this invention are presented herein for the purpose of illustration and description only; it is not intended to be exhaustive or to be limited to the precise form disclosed.

In order to provide a skin-like equivalent, it is necessary to develop a suitable matrix used for skin scaffolds. In order to simulate the composition of extracellular matrix, the present invention selects gelatin, chondroitin 6 sulfate, and hyaluronic acid as basal materials, and uses the lyophilization to fabricate the matrix membrane with bilayer porous structure at different freezing temperatures. The carbodiimide is used as the cross-linking agent to improve the mechanical property of bilayer porous matrix membrane.

According to the method of the present invention, after the bilayer porous matrix is prepared, it can be used to cultivate the skin cell. After it is cultivated for 3 to 4 weeks, the keratinocyte can be differentiated from the basal layer to the suprabasal layer and cornified squamous layer upwards. Meantime, the dermal fibroblasts start to secrete their own extracellular matrix to substitute the original bilayer porous matrix gradually and develop a derma-like structure.

### EMBODIMENT 1: PREPARATION OF THE BILAYER POROUS MATRIX

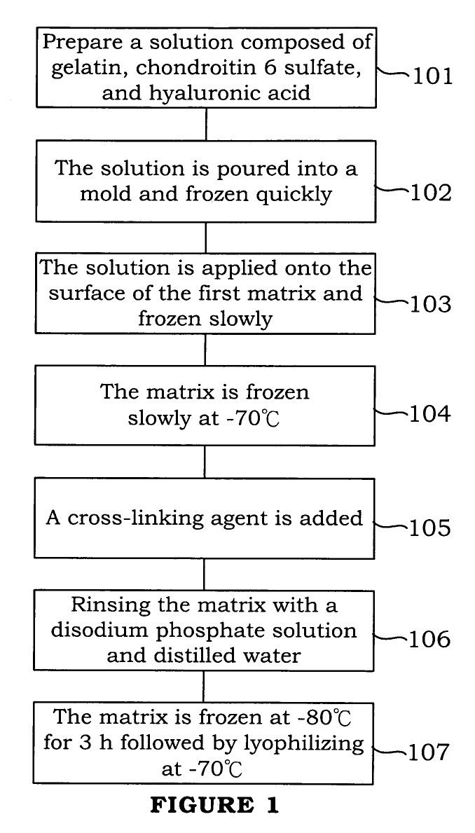

Referring to step 101 shown in FIG. 1, a solution of gelatin, chondroitin 6 sulfate, and hyaluronic acid is prepared first. At room temperature, 5 to 10 wt % of gelatin (Cat. No. G-2500, purchased from Sigma Chemical, USA) is dissolved in distilled water. The powder of chondroitin 6 sulfate (Cat. No. C-4384, purchased from Sigma Chemical, USA) and hyaluronic acid (Cat. No. H-5388, purchased from Sigma Chemical, USA) are added to the gelatin solution with the final concentration of 0.5 to 2.5 wt % of chondroitin 6 sulfate and 0.3 to 0.5 wt % of hyaluronic acid, respectively. The solution is then well mixed at 37° C. for an hour.

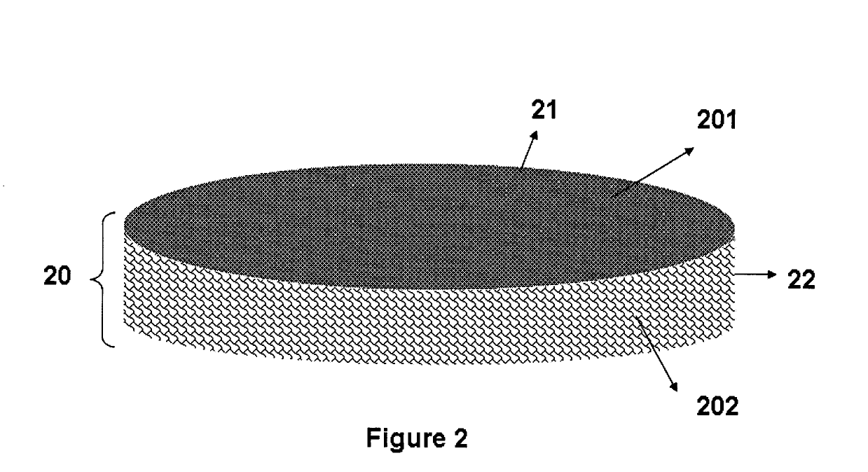

As step 102 shown in FIG. 1, a first layer of the porous matrix is prepared. The 0.5 ml of gelatin, chondroitin 6 sulfate, and hyaluronic acid solution prepared above is poured into a circular stainless mold (1.5 cm in diameter). For the preparation of smaller pore size matrix, the solution/mold is put in liquid nitrogen and frozen quickly to the temperature of −196° C. for 1 to 2 min, to form the first layer of the porous matrix. As the first porous matrix 21 shown in FIG. 2, it possesses a plurality of smaller first pore 201.

As step 103 shown in FIG. 1, a second layer of the porous matrix is prepared. To prepare the second layer of the porous matrix, another 0.5 ml of gelatin, chondroitin 6 sulfate, and hyaluronic acid solution prepared above is applied onto the surface of the first porous matrix 21, and is then frozen at −80° C. for 3 h, to form the second layer of the porous matrix. As the second porous matrix 22 shown in FIG. 2, it possesses a plurality of larger second pore 202. The first porous matrix 21 and the second porous matrix are combined to the bilayer porous matrix 20.

As step 104 shown in FIG. 1, the bilayer porous matrix is prepared. The bilayer porous matrix 20 frozen at −80° C. is taken out and is then frozen at −70° C., preferably for several hours.

As step 105 shown in FIG. 1, add a cross-linking agent to the prepared bilayer porous matrix 20 to react at 4° C. for several hours preferably. The cross-linking agent is the solution of 0.5 to 1 wt % 1-ethyl-3-(3-dimethylaminopropyl) carbodiimide (EDAC) with pH 5.75 and 0.25 wt % N-hydroxysuccinimide.

As step 106 shown in FIG. 1, after the reaction is completed, the prepared bilayer porous matrix 20 is immersed in a disodium phosphate solution, sonicated 5 times in distilled water to remove residual carbodiimide.

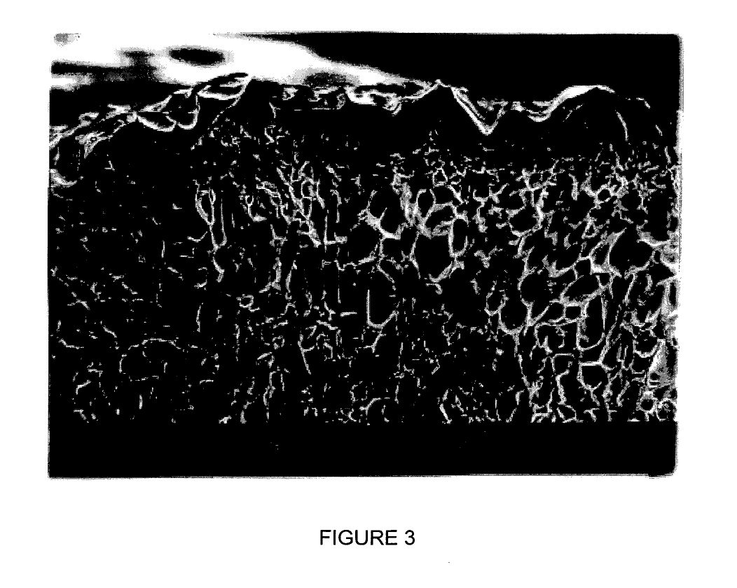

Finally, as step 107 shown in FIG. 1, the rinsed bilayer porous matrix 20 is frozen at −80° C. for 3 h followed by lyophilizing at −70° C. FIG. 3 is the scanning electron microscope image of the prepared bilayer porous matrix, the level and pore size change of porous matrix can be seen clearly.

### EMBODIMENT 2: THE UTILIZATION OF BILAYER POROUS MATRIX FOR CELL CULTURE

The prepared bilayer porous matrix 20 is used as the scaffold for cell culture. The spinner flask is used to cultivate dermal fibroblasts (FB) to achieve the advantages of high cultivation efficiency and uniform cell distribution. In the second porous matrix 22 (lower layer, prepared at −80° C.) of the bilayer porous matrix 20, the pore is larger (75 to 150 μm) and sparse (70 to 80% in porosity), which is suitable for the migration and growth of dermal fibroblasts.

The epidermal keratinocyte (K) is cultivated on the dermal equivalent with cultivated dermal fibroblast. In the first porous matrix 21 (upper layer, prepared at −196° C.), the pore is smaller (20 to 40 μm) and denser (30 to 40% in porosity), which is suitable for the attachment and proliferation of epidermal keratinocytes. The interconnected pores between two layers can provide the interaction opportunities for released cytokines and growth factors by dermal fibroblasts and epidermal keratinocytes, to speed the quick growth and differentiation of skin tissue.

Then, immerse the skin equivalent under the culture medium for some time, and move it to air-liquid interface to mature and differentiate skin tissues. After cultivated for 3 to 4 weeks, the keratinocytes can be differentiated from the basal layer to the suprabasal layer and cornified squamous layer upwards. Meantime, the dermal fibroblasts start to secrete their own extracellular matrix to substitute the original bilayer porous matrix gradually and develop a derma-like structure.

The present invention uses different freezing rates and lyophilization to prepare the matrix with bilayer porous structures, which can be used as the scaffold of artificial skin. The scaffold has good physical-chemical property and biocompatibility. After in vitro culture, the scaffold will be degraded gradually and substituted by new extracellular matrix secreted from the cell. After several weeks, new skin-like tissue structure including epidermal layer and dermal layer can be grown preliminarily. After the animal experiment, it is found that it not only can promote the repair and regeneration of wound, but also can provide suitable mechanical strength to newly grown skin tissue. It will have great potential on clinical application in the future. It can help the patient who requires the skin graft operation for large-area burn, or the wound is difficult to be closed, i.e. diabetes foot ulcers.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A biocompatible bilayer porous matrix, comprising:
a first porous matrix, wherein a pore size of said first porous matrix being between 10 to 50 μm, a matrix thickness of said first porous matrix being between 80 to 120 μm; and
a second porous matrix, wherein a pore size of said second porous matrix being between with 50 to 180 μm, a matrix thickness of said second porous matrix being between 500 to 900 μm; wherein said first porous matrix and said second porous matrix are composed of a cross-linked gelatin, a chondroitin 6 sulfate and a hyaluronic acid.

2. The matrix of claim 1, wherein said pore size of said first porous matrix is between 20 to 40 μm.

3. The matrix of claim 1, wherein said matrix thickness of said first porous matrix is 90 to 110 μm.

4. The matrix of claim 1, wherein said pore size of said second porous matrix is 75 to 150 μm.

5. The matrix of claim 1, wherein said matrix thickness of said second porous matrix is 600 to 800 μm.

6. A method of preparing a biocompatible bilayer porous matrix, comprising:
(a) preparing an aqueous solution of gelatin, chondroitin 6 sulfate, and hyaluronic acid;
(b) pouring said aqueous solution into a mold, and freezing said aqueous solution quickly to form a first porous matrix;
(c) applying said aqueous solution on the surface of said first porous matrix, and freezing slowly to form a second porous matrix; and
(d) adding a cross-linking agent to initiate the cross-linkage of said porous matrixes.

7. The method of claim 6, wherein the preparation of said aqueous solution is to dissolve gelatin in water at room temperature, and then add chondroitin 6 sulfate and hyaluronic acid separately.

8. The method of claim 6, wherein the composition of said aqueous solution is 5 to 10 wt % of gelatin, 0.5 to 2.5 wt % of chondroitin 6 sulfate and 0.3 to 0.5 wt % of hyaluronic acid.

9. The method of claim 6, wherein said first and second porous matrixes are fabricated through different freezing temperatures and time durations to form the pores with different size and density.

10. The method of claim 6, wherein said aqueous solution is frozen quickly to the temperature of −196° C. for 1 to 2 min to form said first porous matrix.

11. The method of claim 6, wherein said aqueous solution is frozen to the temperature of −80° C. for 180 min to form said second porous matrix.

12. The method of claim 6, wherein the step (c) further includes step (cl): lyophilizing said porous matrix at −70° C.

13. The method of claim 6, wherein said cross-linking agent is carbodiimide.

14. The method of claim 13, wherein said cross-linking agent is 0.5 to 1 wt % of 1-ethyl-3-(3-dimethylaminopropyl) carbodiimide (EDAC).

15. The method of claim 14, wherein said cross-linking agent is 0.25 wt % of N-hydroxysuccinimide solution, which is reacted at pH 5.75 and 4° C.

16. The method of claim 6, wherein said porous matrix is rinsed by disodium phosphate solution to remove residual carbodiimide after the cross-linking reaction

17. The method of claim 16, wherein after rinsing, said porous matrix is frozen at −80° C. followed by lyophilizing at −70° C.

18. The method of animal cell culture using said bilayer porous matrix of claim 1, wherein said first and second porous matrix are used to cultivate different cells respectively.

19. The method of claim 18, wherein said first porous matrix is used to cultivate epidermal keratinocytes, and said second porous matrix is used to cultivate dermal fibroblasts.

20. The method of claim 18, wherein said animal cell is a human cell.
